(19) United States
(12) Patent Application Publication
(10) Pub. No.: US 2003/0131845 A1
(43) Pub. Date: Jul. 17, 2003

(54) Auxiliary device for placing larynx cover for anesthetization

(75) Inventor: Bih-Chern Lin, Taichung (TW)

Correspondence Address: Dougherty & Troxell, ONE SKYLINE PLACE, SUITE 1404, 5205 LEESBURG PIKE, FALLS CHURCH, VA, 22041, US

(73) Assignee: Future Top Medical Environment Technic Co., Ltd.

(21) Appl. No.: 10/043,120

(22) Filed: Jan. 14, 2002

(51) Int. Cl. A61M 16/00

(52) U.S. Cl. 128/200.26; 128/207.14

(57) ABSTRACT

An auxiliary device for placing a larynx cover for anesthetization, it comprises a handle, a fixing sleeve, a pushing rod and a soft sleeve. The pushing rod is a flexible and elongate metallic rod, has on an end thereof the soft sleeve, and has on its front end a forked opening and on its bottom a bottom poker; the other end of the pushing rod has a turning portion. When the handle are connected with the pushing rod, the flexible pushing rod is extended into an air inlet pipe of the larynx cover to abut against the bottom of the larynx cover and push it forwardly by means of the bottom poker, so that the forked opening of the soft sleeve abuts on a protrusion on an annular rubber pipe in the larynx cover. The handle and the air inlet pipe are firmly combined by the fixing sleeve, the larynx cover will not be departed from the auxiliary device; and the larynx cover can be in an arciform shape without an opening, it can fit the curvature of the throat and will not poke the root of a tong of a patient. Just remove the fixing sleeve after positioning; the pushing rod can be drawn out in favor of working of a physician or a nurse.

### BACKGROUND OF THE INVENTION

1. Field of the Invention

The present invention is related to an auxiliary device for placing a larynx cover for anesthetization which prevents the larynx cover from curving and makes the latter more sustaining; the latter will not hurt the root of a tong when it is placed in a mouth of a patient, so that a physician or a nurse is convenient to use it; and especially to such an auxiliary device applied in the medical field for patients having been anesthetized or to be anesthetized.

2. Description of the Prior Art

Among modes of anesthetization used in the common medical field, direct injection of anesthetics for patients or applying anesthetic gas to patients is the most usual one. The mode of applying anesthetic gas to patients has to use a larynx cover, such as are shown in FIGS. 1, 2, a cover having a bottom opening, an aerating ring and a slightly tapered end is mounted on one end of an air inlet pipe, the aerating ring is connected with an aerating pipe; the cover as a whole is placed into the throat of a patient, the tapered end of the cover is abutted against the esophagus of the patient, the opening is placed in the trachea of him; air entering the aerating pipe aerates and expands the aerating ring, so that the throat of the patient and the cover do not have a gap created therebetween, and the anesthetic gas entering via the air inlet pipe will have no gap to escape.

The above stated larynx cover can completely render anesthetic gas to get into a human body, however, by virtue that the larynx cover is soft before it is aerated, when it is placed into the throat of the human body, it is supported mostly by the trachea; hence a physician or a nurse does not easily success in placing the larynx cover in, and this is not an ideal case.

In view of the defect resided in the conventional structure, the inventor of the present invention studied to improve and developed an auxiliary device for placing a larynx cover for anesthetization of which the larynx cover is flexible but uneasy to curve and has more sustaining capability. So that medical curing persons without experience of practicing multiple times can easily and successfully use the auxiliary device. And this is exactly the motive of the present invention.

### SUMMARY OF THE INVENTION

The primary object of the present invention is to provide an auxiliary device for placing a larynx cover for anesthetization of which the larynx cover is flexible and has more sustaining capability.

The secondary object of the present invention is to provide an auxiliary device for placing a larynx cover for anesthetization, the larynx cover is not easily departed, and does not hurt the root of a tong of a patient.

Another object of the present invention is to provide an auxiliary device for placing a larynx cover for anesthetization in favor that a physician or a nurse conveniently uses anesthetics.

To achieve the above stated objects, the present invention mainly is comprised of: a handle, a fixing sleeve, a pushing rod and a soft sleeve, the pushing rod is provided on an end thereof with the soft sleeve which is provided on the front end thereof with a forked opening and on the bottom thereof with a bottom poker. The other end of the pushing rod has a turning portion. When the handle and the pushing rod are combined with each other, the flexible pushing rod is extended into the air inlet pipe of the larynx cover to abut against the bottom of the larynx cover and push it forwardly by means of the bottom poker of the soft sleeve, so that the forked opening on the front end of the soft sleeve abuts on a protrusion provided on an annular rubber pipe in the larynx cover. Meantime, the handle and the air inlet pipe are firmly connected by the fixing sleeve, thereby the larynx cover will not be departed from the auxiliary device, and the larynx cover can be in an arciform shape without an opening, and it can fit the curvature of the throat in the throat and will not poke the root of a tong of a patient. After positioning, it needs only to remove the fixing sleeve; then the pushing rod can be drawn out in favor of working of a physician or a nurse.

The present invention will be apparent in its particular structural contents and other characteristics after reading the detailed description of the preferred embodiment thereof in reference to the accompanying drawings.

### BRIEF DESCRIPTION OF THE DRAWINGS

FIGS. 1, 2 are schematic views showing use of a conventional auxiliary device;

FIG. 3 is a perspective view of an embodiment of the present invention;

FIG. 4 is an analytic perspective view showing the elements of the embodiment of the present invention;

FIG. 5 is a schematic view showing use of a larynx cover together with the embodiment of the present invention;

FIG. 6 is a sectional view showing use of the larynx cover together with the embodiment of the present invention;

FIGS. 7-9 are schematic views showing use of the embodiment of the present invention.

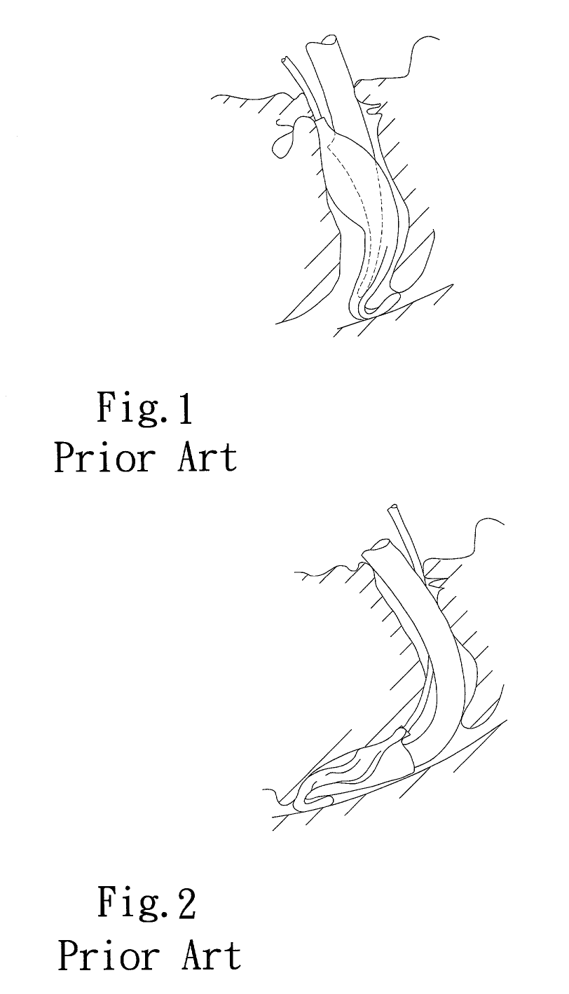

### DETAILED DESCRIPTION OF THE PREFERRED EMBODIMENT

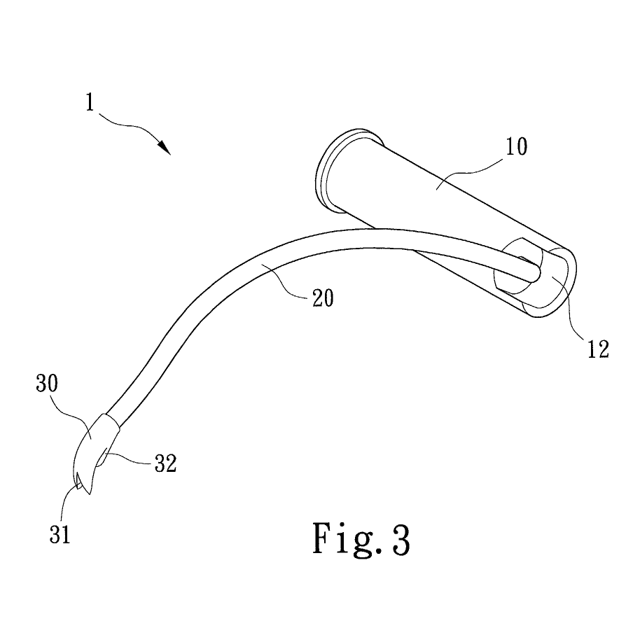

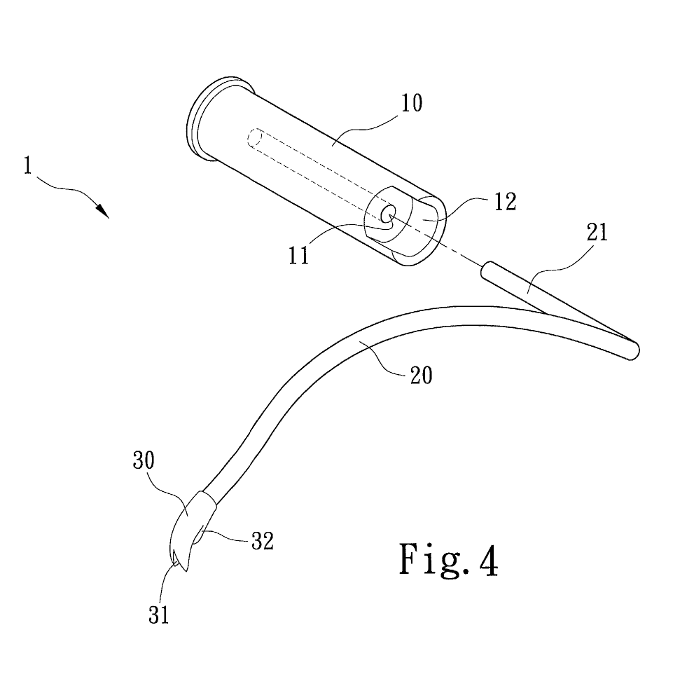

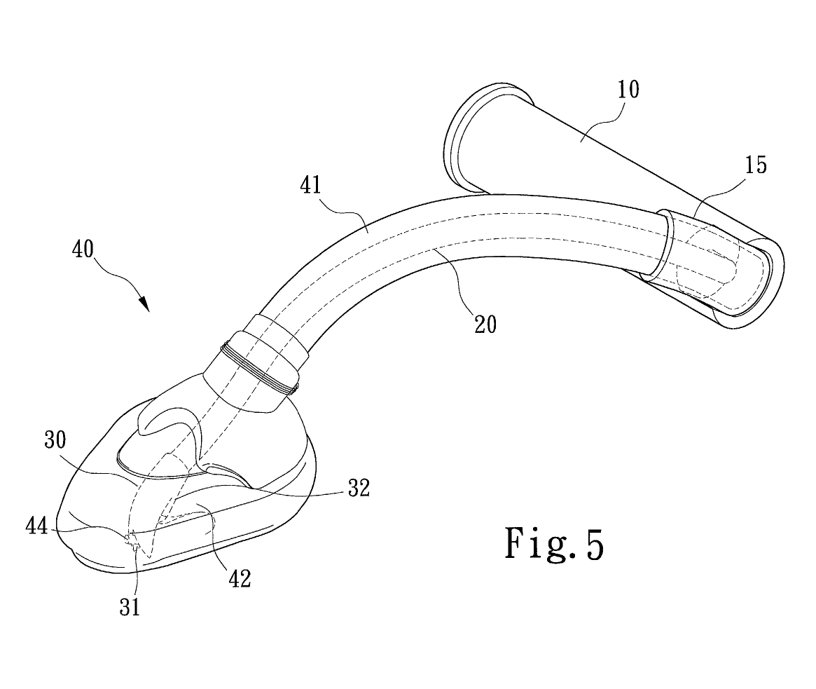

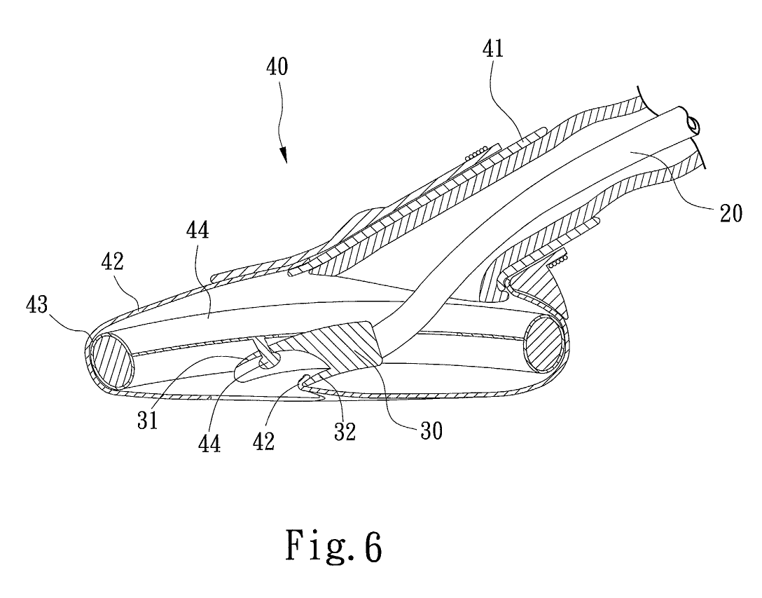

Referring firstly to FIGS. 3, 4, the auxiliary device 1 of the present invention is comprised of a handle 10, a fixing sleeve 15, a pushing rod 20 and a soft sleeve 30. The handle 10 is provided on one side thereof with a notch 12, a hole 11 is provided centrally of the notch 12, the shank of the handle 10 has curved recessed portions for fingers to meet the human body engineering for convenience of holding with a hand. The fixing sleeve 15 is made of rubber. The pushing rod 20 is a flexible and elongate metallic rod, and is provided on an end thereof with a turning portion 21 being bent for 90 degrees, it can be connected with the hole 11 of the handle 10. The pushing rod 20 is provided with the soft sleeve 30 slipped over the front end thereof, which soft sleeve 30 is made of rubber or plastic.

The pushing rod 20 is flexible and can be bent to have any desired radian, while the soft sleeve 30 provided on the front end of the pushing rod 20 has on the front end thereof a forked opening 31 and on the bottom thereof a bottom poker 32. Referring to FIGS. 3 and 4 simultaneously, the soft sleeve 30 envelops the sharper metallic end of the pushing rod 20, so that when it is extended through an inlet pipe 41 of a larynx cover 40, the bottom poker 32 abuts against the bottom portion of a silicone sleeve 42 on the outermost layer of the larynx cover 40 and push forwardly until the forked opening 31 provided on the front end of the soft sleeve 30 abuts on a protrusion 44 provided on an annular rubber pipe 43 in the larynx cover 40. Under the condition that the larynx cover 40 is not to be pierced to broke, and the bottom opening of the larynx cover 40 is reduced to more meet the arciform shape of the throat, the larynx cover 40 thereby is more sustaining, hence medical curing persons without enough experience can easily use the auxiliary device.

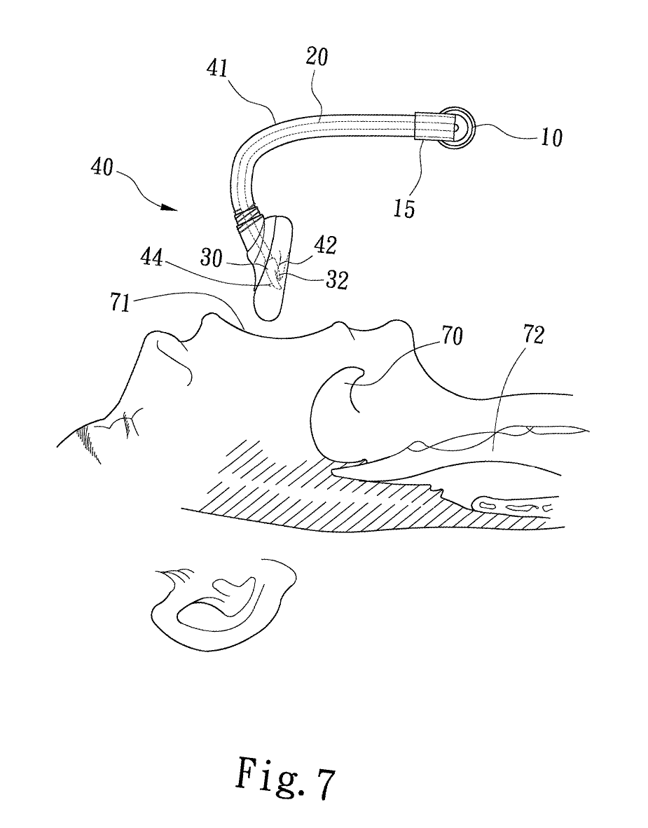

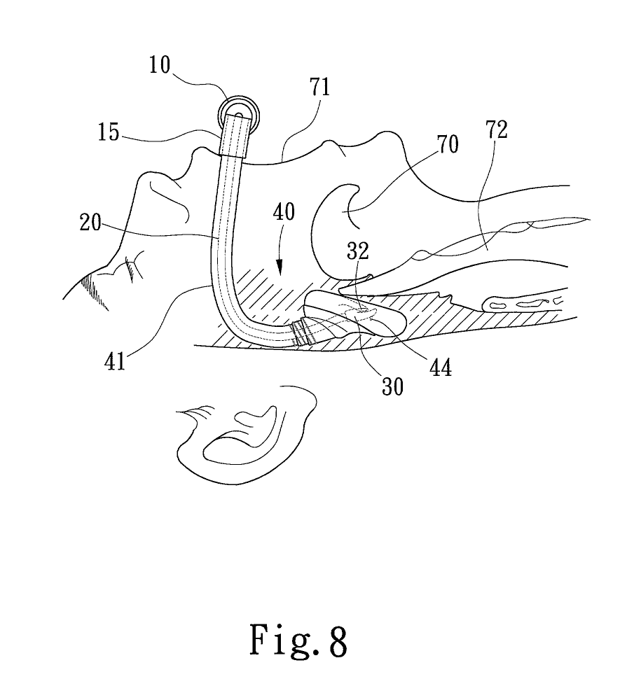

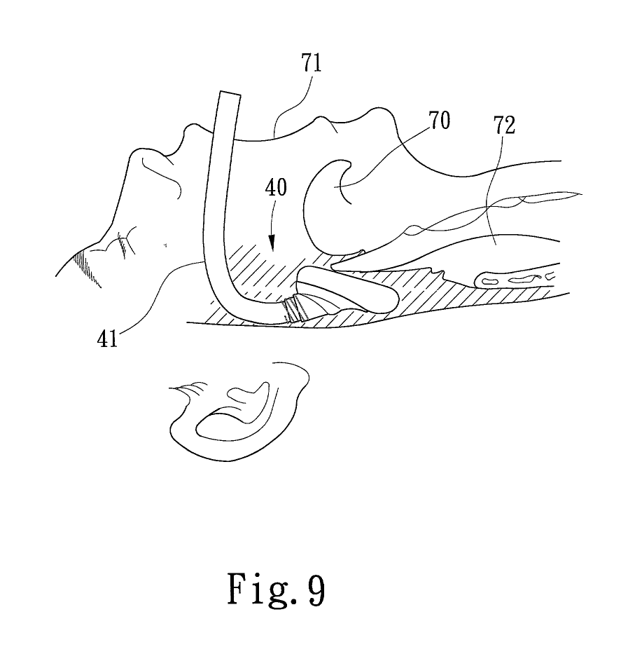

Referring to FIGS. 7-9, the fixing sleeve 15 is firstly slipped over the end of the inlet pipe 41 to increase the diameter of the inlet pipe 41, then the turning portion 21 on one end of the pushing rod 20 is inserted into the hole 11 of the handle 10; and the turning portion 21 is bent to have a desired radian, then is extended through the inlet pipe 41 of the larynx cover 40 to render the bottom poker 32 of the soft sleeve 30 to abut against the bottom portion of a silicone sleeve 42 and to push them forwardly in order that the central forked opening 31 on the front end of the soft sleeve 30 abuts on a protrusion 44 provided on the annular rubber pipe 43 in the larynx cover 40, and to make the bottom opening of the larynx cover 40 reduced. Now the end of the inlet pipe 41 slipped thereover with the fixing sleeve 15 is squeezed into the notch 12 of the handle 10 to tightly fit therewith, thereby, the auxiliary device 1 is firmly connected with the larynx cover 40.

After the above operation, the shape of the larynx cover 40 meets the curvature of the throat; the larynx cover 40 can thereby placed into the oral cavity 71 of a patient. By virtue that the pushing rod 20 has the sustaining ability, the larynx cover 40 with bending curvatures under the action of the pushing rod 20 can be pushed forwardly well along the esophagus. And by reason that the bottom opening of the larynx cover 40 has been reduced, it will not hurt the root 70 of the tong of the patient by scraping, and can arrive the trachea 72 safely. When the operation of placing the larynx cover 40 has been done, it needs only to depart the fixing sleeve 15 from the notch 12 of the handle 10 to directly draw out the auxiliary device 1 rearwardly to render the forked opening 31 on the front end of the soft sleeve 30 to remove from the protrusion 44 provided on the annular rubber pipe 43, and the bottom poker 32 is removed from the bottom portion of a silicone sleeve 42, the bottom opening of the larynx cover 40 is opened again to cover the trachea 72, and the fixing sleeve 15 is removed from the end of the inlet pipe 41 to render the inlet pipe 41 to connect with the gas output port of a anesthetizing machine to complete anesthetization.

The present invention uses the handle for holding by a hand of a person, and has a pushing rod with sustaining capability to hold a soft larynx cover, and has a soft sleeve with a forked opening and a bottom poker on the front end thereof. The bottom poker abuts against the bottom portion of a silicone sleeve and pushes it forwardly in order that the bottom opening of the larynx cover is reduced; it will not hurt the root of the tong of a patient. The auxiliary device thereby is convenient for a physician or a nurse of less experience to use; and has value of practicality.

The above stated is only for illustrating a preferred embodiment of the present invention, and not for giving any limitation to the scope of the present invention. Various modifications or changes can be made to the elements of the present invention without departing from the spirit and principle of this invention, they all fall within the scope of the appended claims and are intended to form part of this invention.

What is claimed is:

1. An auxiliary device for placing a larynx cover for anesthetization, said device is comprised of a pushing rod, a soft sleeve, a handle and a fixing sleeve;
said pushing rod is a flexible and elongate metallic rod, is provided on an end thereof with said soft sleeve, and is provided on the other end thereof with a turning portion;
said soft sleeve is provided on the front end thereof with a forked opening and on the bottom thereof with a bottom poker;
said handle is provided on one side thereof with a notch, a hole is provided centrally of said notch; and
said fixing sleeve is made of rubber;
when said handle and said pushing rod are connected with each other, said flexible pushing rod is extended into an air inlet pipe of said larynx cover to abut against the bottom of said larynx cover and push it forwardly by means of said bottom poker of said soft sleeve, so that said forked opening on said front end of said soft sleeve abuts on a protrusion provided on an annular rubber pipe in said larynx cover, thereby said larynx cover is in an arciform shape without an opening in favor of operating of a physician or a nurse.

2. An auxiliary device for placing a larynx cover for anesthetization as in claim 1, wherein,
the shank of said handle has curved recessed portions for fingers to meet the human body engineering.

3. An auxiliary device for placing a larynx cover for anesthetization as in claim 1, wherein,
said fixing sleeve made of rubber is sleeve with tight binding action.
